(19) United States
(12) Patent Application Publication
(10) Pub. No.: US 2023/0381263 A1
(43) Pub. Date: Nov. 30, 2023

(54) MULTICOMPONENT COMPOSITION AND USE THEREOF IN THE TREATMENT OF PROSTATE DISEASES

(71) Applicant: KOLINPHARMA S.P.A., Milano (MI) (IT)

(72) Inventors: Rita Paola PETRELLI, Milano (MI) (IT); Valeria CURTI, Milano (MI) (IT)

(21) Appl. No.: 18/032,779

(22) PCT Filed: Nov. 16, 2021
(86) PCT No.: PCT/IB2021/060617

§ 371 Date: Apr. 19, 2023

(30) Foreign Application Priority Data
Nov. 16, 2020 (IT) 102020000027423

(51) Int. Cl. A61K 36/899; A61K 31/352; A61K 31/385; A61K 31/405; A61K 9/00; A61P 13/08

(52) U.S. Cl. CPC A61K 36/899; A61K 31/352; A61K 31/385; A61K 31/405; A61K 9/0053; A61P 13/08

(57) ABSTRACT

The present invention relates to multicomponent compositions comprising a Secale cereale (L.) pollen extract (Graminex®), quercetin, lipoic acid, and tryptophan and/or a saffron extract, wherein said compositions are capable of preventively and/or curatively treating prostate diseases and/or symptoms, in particular nonbacterial prostatitis/chronic pelvic pain syndrome (CP/CPPS) and/or benign prostatic hyperplasia (BPH).

The present invention relates to multicomponent compositions comprising a Secale cereale (L.) pollen extract (for example Graminex®), quercetin, lipoic acid, and tryptophan and/or a saffron extract, wherein said compositions are capable of preventively and/or curatively treating prostate diseases and/or symptoms, in particular nonbacterial prostatitis/chronic pelvic pain syndrome (CP/CPPS) and/or benign prostatic hyperplasia (BPH).

The prostate is a glandular organ of the male genital system which develops around the initial portion of the urethra and it is passed through by the two ejaculatory ducts, it is cone-shaped and it is located between the medial margins of the two elevator muscles of the anus.

Diseases or related symptoms of the prostate are a public health problem with a significant impact from the epidemiological, clinical and economic point of view. 35% to 50% of men report that they have had symptoms that can be linked to prostatitis during their lifetime.

According to the National Institutes of Health (NIH), prostatitis are divided into 4 categories.

The first category (the least frequent) includes acute bacterial prostatitis, characterised by the presence of bacteria (mainly Gram-negative, for example Escherichia coli) and acute infection of the prostate gland The second category includes chronic bacterial prostatitis (mainly Gram-negative, for example Escherichia coli). These are chronic (persistence for at least 3 months) or recurrent infections of the prostate with bacterial aetiology, characterised by painful urinary and sexual symptoms.

The third category is the chronic nonbacterial prostatitis/chronic pelvic pain syndrome (CP/CPPS) and it is the most common category of prostatitis in adult men. This Class Ill can be further divided into inflammatory (IIIa) and non-inflammatory (IIIb). Clinical symptoms and manifestations comprise pelvic or perineal (lower abdomen, testicles, penis) pain in the absence of pathogenic bacteria in prostatic secretions and difficulty in emptying (including irritative and obstructive symptoms). A very important aspect of this type of disorders is the decline in quality of life, which can lead to depression. The aetiology to date remains unknown and there appears to be no infection at the root of the problem. Potential causes could be the presence of inflammation due to trauma, autoimmunity, reaction to normal prostate flora, neurogenic pain, increased prostate tissue pressure and interaction of somatic and psychological factors. Psychological stress, including anxiety and fear of serious diseases, appears to be common in men with CP/CPPS symptoms and it may be a contributing factor to the onset and development of the disease. As regards treatment, several non-pharmacological drugs and therapies, including physical therapy and psychological support are available. There is no uniformly accepted treatment regimen and many treatments are used combined. Pharmacological therapies include: alpha-blockers, antibiotics, anti-inflammatories, 5-alpha reductase inhibitors and for neuropathic pain drugs.

The fourth category includes asymptomatic inflammatory prostatitis which does not require treatment.

Furthermore, a relevant prostate disease is benign prostatic hyperplasia (BPH). Benign prostatic hyperplasia (BPH) is a chronic condition associated with the lower urinary tract symptoms (LUTS) and it affects nearly 3 out of 4 men in the seventh decade of life. Though the mechanism which leads to the onset of BPH is not entirely clear, several assumptions have been made involving metabolic aspects, hormonal aspects and inflammatory processes. Systemic and localised inflammation, same case applying chronic inflammation, are associated with LUTS/BPH. With regard to metabolic syndrome, scientific evidence points out that this is related to lower urinary tract symptoms (LUTS). The expression metabolic syndrome is used to indicate a condition characterised by the concurrent presence of: obesity, with abdominal fat deposition (waistline in men >102 cm), hypertiglyceridemia (>150 mg/dl), HDL cholesterol values below 40 mg/dl (in men), arterial hypertension (values above 130/85 mmHg) and high fasting blood glucose levels (>110 mg/dl). It has been demonstrated that patients with one or more of these symptoms show a faster progression to BHP, for example high fasting insulin levels. At the anatomical level, enlarged prostate results in compression of the urethra, leading to an increased resistance of urinary stream at the bladder outlet. The most widely used symptom assessment score is IPSS, which is based on the classification of seven symptoms (weak stream, urinary hesitation, intermittency, incomplete emptying, straining, urgency, frequency, nocturia). The pharmacological treatment of BHP provides for an initial monotherapy based on alpha-1-adrenergic antagonists, 5-alpha-reductase inhibitors, anticholinergic agents and 5-phosphodiesterase inhibitors, and a combination thereof in the most severe cases.

The most common problems among those mentioned above, and those which further complicate patient management, are chronic nonbacterial prostatitis/chronic pelvic pain syndrome (CP/CPPS) and benign prostatic hyperplasia (BHP). In both cases, patients report lower urinary tract symptoms (LUTS). Prostate inflammation is considered a major factor in determining both prostate growth and progression of symptoms. Besides increase in the concentrations of reactive oxygen species and nitrogen reactive species (ROS/RNS), the common aspects of inflammation are due to the activation of astrocytes and microglia, the involvement of the immune system, with the resulting hyper-expression of pro-inflammatory cytokines. Clinical observations suggest that chronic inflammation correlates CP/CPPS and BPH.

Furthermore, the decline in quality of life in these patients results in the manifestation of depression.

Therefore, there is felt the need to provide effective and side effect-free compositions for use in a method for the preventive and/or curative treatment of prostate diseases and/or symptoms, in particular nonbacterial prostatitis/chronic pelvic pain syndrome (CP/CPPS) and/or benign prostatic hyperplasia (BPH). Even though compositions for the treatment and/or prevention of prostate dysfunctions are available on the market, often these treatments are not effective or they are only partially effective.

For example, WO 2020/148224 A1 relates to a composition comprising a pollen extract to be used in the treatment and/or in the prevention of lower urinary tract symptoms (LUTS) mainly associated with the filling, urination and/or post-urination phases in women.

KR20120058656A discloses a pharmaceutical composition containing a natural extract and alpha blockers for suppressing 5-alpha reductase and for preventing and treating prostate disorder. Natural extracts can be selected from the extracts of saw palmetto, nettle, pygeum, pollen and pumpkin seeds.

EP 2 510 931 A1 discloses a composition for the treatment and prevention of adenoma and prostatitis, comprising pollen, ascorbic acid, vitamin E and excipients.

US 2001/025059 A1 discloses a composition and method for the treatment of prostate-related dysfunctions and, in particular, of nonbacterial prostatitis and, even more particularly, of chronic nonbacterial prostatitis. The composition is mainly based on the use of a bioflavonoid.

However, none of these documents discloses a composition comprising (a) a Secale cereale (L.) pollen extract, (b) a quercetin, (c) a lipoic acid or an acceptable pharmaceutical or food grade salt thereof, and (d) a tryptophan. Just like they do not disclose the synergistic effect of these active components in the preventive and/or curative treatment of a prostate disease or symptom.

Therefore, in order to treat said prostate diseases and related symptoms, in particular chronic nonbacterial prostatitis/chronic pelvic pain syndrome (CP/CPPS) and benign prostatic hyperplasia (BHP), therefore there arises the need to inhibit inflammation and oxidative stress, as well as to act at the mood level, resulting in raising the same.

The technical problem addressed and solved by the present invention lies in providing effective and side effect-free compositions for use in a method for the preventive and/or curative treatment of prostate diseases and/or symptoms, in particular nonbacterial prostatitis/chronic pelvic pain syndrome (CP/CPPS) and/or benign prostatic hyperplasia (BPH).

In order to overcome said technical problems, following an intense research phase, the Applicant provides multicomponent mixtures or compositions (mixtures or compositions of the invention) comprising (a) a Secale cereale (L.) pollen extract pollen extract (for example the product under the trade name Graminex® G63®) (in short hereinafter “pollen extract”), (b) a quercetin or a plant extract titrated in quercetin (for example Sophora japonica), (c) a lipoic acid, and (d) a tryptophan and/or a compound having mood-modulating activity similar to tryptophan (for example, a saffron extract), as reported in the present description and in the attached claims.

When administered to a subject in need, said mixtures or compositions of the invention are capable of preventing and/or treating prostate diseases and/or symptoms, in particular nonbacterial prostatitis/chronic pelvic pain syndrome (CP/CPPS) and/or benign prostatic hyperplasia (BPH), in an effective, rapid and side effect-free manner.

Said preventive and/or curative treatment activity of the mixtures or compositions of the invention is due to the specific and innovative combination of the active components (as defined in the present description) which confers an immunostimulatory, anti-inflammatory, antioxidant and/or mood-modulating action/activity thereto, making the mixtures or composition of the invention particularly effective.

For example, pollen extract (Graminex®) has an anti-inflammatory, antioxidant and anti-pain effect. It has been demonstrated that the Graminex® G63 pollen extract has a protective and antioxidant effect in human prostate cancer cells (PC3) and it inhibits the production of H₂O₂-induced ROS. Furthermore, Graminex® G63 pollen extract has anti-inflammatory, anti-pain and antioxidant properties on prostate tissue samples. Said anti-inflammatory activity can be linked to both fractions that the Graminex® G63 pollen extract consists of: liposoluble and water-soluble. Specifically, the liposoluble fraction is capable of inhibiting the biosynthesis of prostaglandins derived from arachidonic acid to the same extent as with nonsteroidal anti-inflammatory agents and cyclooxygenase inhibitors. Furthermore, the water-soluble fraction almost fully inhibits the activity of 5-lipoxygenase and, as a result, the synthesis of leukotrienes. Lastly, a study conducted on an animal model (rat) of 17-β-estradiol-induced nonbacterial prostatitis has demonstrated that pollen extract is capable of reducing IL-6 and TNF-α levels and combating changes induced by the disease at histological level, including acinar glandular inflammation and stromal proliferation.

Queroetin exerts an anti-inflammatory action by inhibiting various inflammatory pathways (for example TNF-α, IL-8 and IL-1a). Furthermore, quercetin is capable of inhibiting enzymes directly involved in inflammation such as lipoxygenases (LOX) and cyclooxygenases (COX), and at the same time inhibiting the deposition of metalkoproteinases (MMPs) in the extracellular matrix, protecting the tissue from leukocyte invasion. Furthermore, animal models and clinical studies have demonstrated the efficacy of quercetin in the protection action against nonbacterial prostatitis/chronic pelvic pain syndrome (CP/CPPS) and in the treatment of patients with chronic pelvic pain.

Lipoic acid (or α-lipoic acid) is a molecule with an antioxidant action. The oxidised form (LA) and reduced (DHLA) form represent a potent redox pair and they can act as ROS scavengers and regenerate endogenous antioxidant levels, such as vitamin C, vitamin E and glutathione. Both forms of the acid can neutralise various types of ROS and form complexes with various metals, depending on affinity, thus acting as chelating agents. The antioxidant properties also confer a neuroprotective action to alpha-lipoic acid. Said neuroprotective action of alpha-lipoic acid is important in the light of the fact that chronic prostatitis or chronic pelvic pain syndromes, at least in some patients, can be initially caused by problems affecting the genitourinary tract (infections, trauma, dysfunctional emptying) which cause inflammation and/or neurogenic damage to the prostate, muscles, tendons and nerves of the pelvis and perineum in anatomically or genetically predisposed men. This series of events can lead to the sensitisation of both the peripheral and central nervous systems, which may ultimately result in pain. Furthermore, said neuroprotective action of the alpha-lipoic acid is of interest for the treatment of neuropathies associated with diabetes, given that various studies report the association between obesity (metabolic syndrome) and benign prostatic hyperplasia.

Lastly, tryptophan acts on mood by increasing levels of serotonin (5-HT) regarding which it is the precursor of synthesis. Furthermore, tryptophan acts on homeostatic sleep regulation, which can be linked to increase in levels of serotonin, the precursor of melatonin, as the hormone responsible for sleep-wake rhythm.

Showing a high safety profile, the mixtures and compositions of the invention can be used by a broad category of subjects, such as adults, the elderly and sportsmen.

The mixtures and compositions of the invention are easy to prepare and cost-effective.

These and other objects which will be apparent from the detailed description that follows are achieved by the mixtures and the compositions the present invention thanks to the technical characteristics present in the description and in the attached claims.

### SUMMARY OF THE INVENTION

A first aspect of the present invention relates to a mixture (in short, mixture of the invention) comprising (a) a Secale cereale (L.) pollen extract (Graminex®), (b) a quercetin, (c) a lipoic acid, and (d) tryptophan and/or a compound having a mood-modulating activity similar to tryptophan (for example, a saffron extract).

A second aspect of the present invention relates to a composition (in short, composition of the invention) comprising said mixture of the invention and at least one acceptable pharmaceutical or food grade additive and/or excipient.

A third aspect of the present invention relates to said mixture or composition of the invention for use as medicament.

A fourth aspect of the present invention relates to said mixture or composition of the invention for use in a method for the preventive and/or curative treatment of prostate diseases and/or symptoms, in particular, nonbacterial prostatitis/chronic pelvic pain syndrome (CP/CPPS) and/or benign prostatic hyperplasia (BPH), in a subject in need, by administering a therapeutically effective amount of the mixture or composition of the present invention to said subject.

### BRIEF DESCRIPTION OF THE FIGURES

FIG. 1: Effect of the active components on the cell viability of PC3 cells. Minimum 4 replicates/experimental group. The data are expressed with respect to the mean of control group C (untreated).

FIG. 2: Effect of the mixture according to the present invention, at different concentrations, on the cell viability of PC3 cells. Minimum 4 replicates/experimental group. The data are expressed with respect to the mean of control group C (untreated).

FIG. 3: Effect of the active components and of the combination thereof on the production of ROS under baseline conditions in PC3 cells. Minimum 4 replicates/experimental group. The data are expressed with respect to the mean of control group C (untreated).

FIG. 4: Effect of the active components and of the combination thereof on the cell viability of PC3 cells in the presence of LPS. Minimum 4 replicates/experimental group. The data are expressed with respect to the mean of control group C (treated with LPS only).

FIGS. 5a and 5b: Effect of the active components and of the combination thereof on the production of IL6 under baseline conditions and induced by LPS in PC3 cells. ***p<0.001 vs. LPS. Minimum 4 replicates/experimental group. The data are expressed with respect to the mean of control group C (untreated).

FIG. 6: Inhibitory effect of the active components and of the combination thereof on the production of LPS-induced IL6 in PC3 cells. *p<0.05; ***p<0.001; vs LPS. Minimum 4 replicates/experimental group. The data are expressed with respect to the mean of control group LPS (treated with the latter only).

### DETAILED DESCRIPTION OF THE INVENTION

Forming an object of the present invention is a mixture comprising or, alternatively, consisting of: (a) a Secale cereale (L.; or rye) pollen extract (for example the product under the trade name Graminex® G63®), (b) a quercetin or a plant extract titrated in queroetin (for example Sophora japonica), (c) a lipoic acid, and (d) a tryptophan.

In the context of the present invention, the expression “queroetin” is used to indicate both queroetin as such (with various degrees of purity, for example from 70% to 99.9% weight/weight) or, alternatively, a plant extract (botanical) comprising (or titrated, according to methods and equipment known to the person skilled in the art) quercetin (for example, titrated from 50% to 99.9% weight/weight).

Alternatively, said mixture of the invention may comprise or, alternatively, consist of: (a) a Secale cereale (L.; or rye) pollen extract (for example Graminex® G63®), (b) a queroetin, (c) a lipoic acid, and (d) tryptophan and/or a compound having mood-modulating activity similar to tryptophan (for example, saffron extract).

Said (a) rye pollen extract (comprised in the mixture of the invention together with (b), (c) and (d)) may comprise or, alternatively, consist of an extract of: rye (Secale cereale L.) pollen, corn (Zea mays L.) pollen and Timothy (Phleum pratense L.) pollen

Said (a) rye pollen extract (comprised in the mixture of the invention together with (b), (c) and (d)), advantageously comprises a water-soluble fraction (soluble in water) and a liposoluble fraction (insoluble in water) in a (water-soluble:liposoluble) weight ratio comprised in the range from 30:1 to 10:1, preferably in a weight ratio of about 20:1 (for example 20±2:1±0.1), wherein the water-soluble fraction is standardised in amino acids and the liposoluble fraction is standardised in phytosterols.

Preferably, said (a) extract (or dry extract) of rye (Secale cereale L.) pollen comprises or, alternatively, consists of the product under the trade name Graminex® G63® (or Flower Pollen Extract™, produced by Graminex® L.L.C., USA).

The product under the trade name Graminex® G63® comprises or, alternatively, consists of a Secale cereale pollen extract.

Alternatively, said product under the trade name Graminex® G63® may comprise or, alternatively, consist of an extract of rye (Secale cereale L.) pollen, corn (Zea mays L.) pollen and/or Timothy (Phleum pratense L.) pollen.

Said product under the trade name Graminex® G63® comprises a standardised extract of rye (Secale cereale L.) pollen and, optionally, corn (Zea mays L.) pollen and Timothy (Phleum pratense L.) pollen (in short, pollen extract), in the form of a powder comprising a water-soluble fraction (soluble in water) and a liposoluble fraction (insoluble in water) in a weight ratio of about 20:1 (hydro:lipo=about 20:1 or 20±2:1±0.1), wherein the water-soluble fraction is standardised in amino acids and the liposoluble fraction is standardised in phytosterols.

In the context of the present invention, the expression “phylosterols” is used to indicate a group of sterols present in the Secale cereale plants and, optionally, Zea mays and Phleum pratense plants. The main phytosterols are: cholesterol, β-sitosterol, campesterol, stigmasterol, brassicasterol, delta-5-avenasterol, cholestanol, sitostanol, campestanol, erythrodiol, uvaol, betulin, clersterol, 24-methylene cholesterol, delta-5-24-stigmastadienol, delta-7-stigmastenol, delta-7-avenasterol, delta-7-campesterol, delta-5-23-stigmastadienol.

An example of the product under the trade name Graminex® has the following characteristics: water-soluble fraction:liposoluble fraction in a 20:1 weight ratio, density (bulk density) 0.30-0.70 g/cc (reference USP 616), pH 3.5-5.8 (reference USP 791), loss on drying: not higher than 6.0% (reference GM 078); particle size: not lower than 95% through 80 mesh (reference GM 075); furthermore, it comprises the following additives/excipients: maltodextrin, microcrystalline cellulose, silicon dioxide, calcium stearate, monocalcium phosphate and gum arabic.

For example, said (a) pollen extract (i.e. Graminex® G63®) comprised in the mixture of the present invention (together with (b), (c) and (d)) has an alpha-amino acid content not lower than about 3.6 mg/250 mg (such as from 3.6 mg to 200 mg, for example 5 mg, 10 mg, 20 mg, 30 mg, 40 mg, 50 mg, 60 mg, 70 mg, 80 mg, 90 mg, 100 mg, 120 mg, 140 mg, 160 mg, 180 mg, on 250 mg) and phytosterols not lower than about 0.2 mg/250 mg (such as from 0.2 mg to 10 mg, for example 0.5 mg, 1 mg, 2 mg, 3 mg, 4 mg, 5 mg, 6 mg, 7 mg, 8 mg or 9 mg, on 250 mg).

The amino acid and/or phytosterol content in said pollen extract may be determined by means of titration methods (for example, titration by means of spectrometry) and standard equipment known to the person skilled in the art.

Said Graminex® (i.e. pollen extract of Secale cereale and, optionally, of Zea mays and Phleum pratense) is obtained by extracting—from rye (and optionally corn and/or Timothy) at the same time—the soluble part (G60®, by extracting with water) and the lipophilic part (GFX (or NAX™), by extracting with carbon dioxide), and by mixing two products deriving from two extractions to obtain the product G63 comprising a combination G600®:GFX=about 20:1, as by weight ratio.

The expression “dry extract” in the context of the present invention is used to indicate an extract in powdered form having a water content in a percentage by weight from 0.05% to 15% (for example, 0.1%, 0.5%, 1%, 2%, 3%, 4%, 5%, 6%, 7%, 8%, 9%, 10%, 11%, 12%, 13%, or 14%), preferably from 0.05% to 10% or from 0.05% to 5%.

Secale cereale (L., 1753), also known as rye, is a cereal widespread in temperate region; scientific classification: kingdom Plantae, subkingdom Tracheobionta, superdivision Spermatophyta, division Magnoliophyta, class Liliopsida, order Cyperales, family Poaoeae, genus Secale.

Zea mays (L., 1753), also known as corn, is an annual herbaceous plant of the family of Poaceae; scientific classification: kingdom Plantae, division Magnoiophyta, class Liliopsida, order Poales, family Poaceae, subfamily Panicoideae, tribe Andropogoneae, genus Zea.

Phleum pratense (L., 1753), also known as Timothy, meadow cat's-tail or timothy-grass, is a plant of the family of Poaceae; scientific classification: kingdom Plantae, subkingdom Tracheobionta, division Magnoliophyta, class Liliopsida, order Cyperales, family Poaceae, subfamily Pooideae, tribe Aveneae, genus Phleum.

According to a preferred embodiment, said mixture of the invention comprises or, alternatively, consists of: (a) a Secale cereale pollen extract (and, optionally, of Zea mays and Phleum pratense) comprising a water-soluble fraction and a liposoluble fraction in a by weight ratio from 30:1 to 10:1, preferably about 20:1, wherein the water-soluble fraction is standardised in amino acids and the liposoluble fraction is standardised in phytosterols (i.e. Graminex® G63®), (b) quercetin, (c) lipoic acid, and (d) tryptophan.

Queroetin (IUPAC name 3,3′,4′,5,7-pentahydroxyflavone, example of CAS No. 6151-25-3) is a flavonoid. An example of quercetin which can be used in the composition of the present invention is queroetin in the form of a Saphora japonica extract, obtained according to methods and equipment known to the person skilled in the art.

Lipoic acid (or alpha-lipoic acid, IUPAC name (R)-5-(1,2-dithiolan-3-yl)pentanoic acid) is a small amphipathic molecule (brute formula C₈W₁₄O₂S₂); example of CAS No. 1077-28-7. In nature it exists in two forms, as cyclic disulfide (oxidised form) or as an open chain under the name of dihydrolipoic acid, which shows two sulfhydryl groups in position 6 and 8; the two forms are easily interconvertible by means of redox reactions.

Tryptophan (IUPAC name L-tryptophan) is an essential amino acid, that is to say that the human organism is not able to synthesise it and must be obtained from foodstuffs or external sources.

Forming an object of the present invention is a composition comprising the mixture of the present invention comprising or, alternatively, consisting of (a), (b), (c) and (d) (as defined in the present description) and at least one acceptable pharmaceutical or food grade additive and/or excipient.

The mixtures or the compositions of the invention are advantageously formulated for oral (or sublingual) administration.

The dosage form of the mixture or composition of the invention may be a solid form, such as tablet, chewable tablet, effervescent tablet, capsule, soft capsule, lozenge, granules or powder (granules or powder to be dissolved in water or mouth dissolvable), or a semi-solid form, such as soft-gel, or a liquid form, such as solution, suspension, dispersion, emulsion or syrup; preferably the composition of the invention is in solid form for oral use, more preferably in tablet form.

The mixture or composition of the invention may advantageously comprise—per “dosing unit” or “daily dose unit”, preferably in solid form (e.g. of one or more tablets, capsules or granules/powder dosed in sachet)—the following amounts:
(a) Secale cereale pollen extract, preferably it comprising [hydrophilic fraction:lipophiic fraction] in a by weight ratio from 30:1 to 11, preferably about 20:1, in an amount comprised from 50 mg to 2000 mg (for example, 100 mg, 200 mg, 300 mg, 400 mg, 500 mg, 600 mg, 700 mg, 800 mg, 1000 mg, 1200 mg, 1400 mg, 1600 mg, or 1800 mg), preferably from 500 mg to 1000 mg (for example, 1000 mg),
(b) quercetin in an amount comprised from 10 mg to 400 mg (for example, 20 mg, 40 mg, 60 mg, 70 mg, 80 mg, 100 mg, 120 mg, 140 mg, 160 mg, 180 mg, 200 mg, 250 mg, 300 mg, or 350 mg), preferably from 100 mg to 200 mg (for example, 200 mg), considering that 200 mg/day is the maximum daily dose per adult subject allowed to date by Italian legislation;
(c) lipoic acid (or a salt thereof) in an amount comprised from 50 mg to 1000 mg (for example 100 mg, 200 mg, 250 mg, 300 mg, 350 mg, 400 mg, 450 mg, 500 mg, 550 mg, 600 mg, 700 mg, 800 mg, or 900 mg), preferably comprised from 250 mg to 600 mg (for example about 600 mg);
(d) tryptophan in an amount comprised from 10 mg to 600 mg (for example, 50 mg, 100 mg, 150 mg, 180 mg, 200 mg, 220 mg, 250 mg, 280 mg, 300 mg, 400 mg, 450 mg, 500 mg, or 550 mg), preferably from 150 mg to 300 mg (for example about 300 mg).

Said “daily dose unit” of the composition of the invention may be administered to a subject in need over the 24-hour interval by means of a single dose or divided into 2, 3 or 4 doses at 4-hour to 12-hour intervals, depending on the type of dosage form and the needs of the subject.

Said “dosing unit” of the composition of the invention may be administered to a subject in need once or twice a day (for example, in the form of a tablet away from meals). Advantageously, said “dosing unit” is administered twice a day in the initial stage of the treatment of the disease or symptom, and once a day in the maintenance stage of the treatment, as the stage subsequent to said initial stage.

The mixture of the invention, such as the mixture of the active ingredients only, may advantageously comprise—per “daily dose unit” or “dosing unit” preferably in solid form (e.g. of one or more tablets, capsules or granules/powder dosed in sachet)—the following percentages by weight with respect to the total weight of the mixture:
(a) Secale cereale pollen extract, preferably it comprising [hydrophilic fraction: lipophiic fraction] in a by weight ratio from 30:1 to 10:1, preferably about 20:1, in an amount comprised from 20% to 80% (for example, 30%, 35%, 40%, 45%, 50%, 55%, 60%, 65%, 70%, or 75%), preferably from 40% to 60% (for example about 45-50%) (for example Graminex®),
(b) quercetin in an amount comprised from 1% to 30% (for example, 2%, 5%, 6%, 7%, 8%, 9%, 10%, 15%, 20%, or 25%), preferably from 5% to 15% (for example about 5%-8%, or about 9-10%);
(c) lipoic acid (or a salt thereof) in an amount comprised from 5% to 60% (for example, 10%, 15%, 20%, 25%, 30%, 35%, 40%, 45%, 50%, or 55%), preferably from 10% to 40% (for example about 28-30%);
(d) tryptophan in an amount comprised from 1% to 50% (for example, 6%, 7%, 8%, 9%, 10%, 15%, 20%, 25%, 30%, 35%, 40%, or 45%), preferably from 5% to 30% (for example about 7%-9%, or about 14-15%).

Forming an object of the present invention is the mixture or composition of the invention (comprising (a), (b), (c), and (d), according to any one of the described embodiments or aspects) for use as medicament.

Forming an object of the present invention is the mixture or composition of the invention (comprising (a), (b), (c), and (d), according to any one of the described embodiments or aspects) for use in a method for the preventive and/or curative treatment of prostate diseases and/or symptoms, preferably wherein said prostate diseases and/or symptoms are selected from nonbacterial prostatitis/chronic pelvic pain syndrome (CP/CPPS), benign prostatic hyperplasia (BPH), and symptoms and/or disorders related with said nonbacterial prostatitis/chronic pelvic pain syndrome (CP/CPPS) and benign prostatic hyperplasia (BPH), in a subject in need, by administering a therapeutically effective amount of the mixture or composition of the present invention to said subject.

The expressions symptoms and/or disorders related with said nonbacterial prostatitis/chronic pelvic pain syndrome (CP/CPPS) and benign prostatic hyperplasia (BPH) are used to indicate, for example constant pain in the scrotum and in the anus or in the central-lower abdomen; pain during or after urination; pain during or after ejaculation; frequent urination, both during the day (pollakiuria) and at night (nocturia); feeling of incomplete bladder emptying after urination; urgency and impossibility of delaying urination; erectile dysfunction and decreased sexual desire, and others known to the person skilled in the art.

The mixture or the composition of the invention may be for use in a method for the preventive or curative treatment of prostate diseases and/or symptoms (as defined above) both when administered to a subject as a single therapy and when administered as an adjuvant of at least one other therapy or composition capable of treating said diseases and/or symptoms.

Forming an object of the present invention is a method for the preventive and/or curative treatment of prostate diseases and/or symptoms, as described above, in a subject in need by administering a therapeutically effective amount of the mixture or composition of the present invention to said subject.

Forming an object of the present invention is the use—for non-medical (non-therapeutic) treatment—of conditions or sensations in a subject related with prostate problems, by administering an appropriate amount of the mixture or composition of the present invention to said subject.

For the sake of clarity, in order to achieve the object of the present invention, the active compounds of the mixture or composition of the present invention as ((a), (b), (c), and (d)) may also be administered separately or in groups (preferably in a time interval of 30 minutes-2-3 hours) and in any order.

Said at least one pharmaceutical or food grade additive and/or excipient, comprised in the composition of the invention together with the mixture of (a), (b), (c), and (d), consists of a substance devoid of therapeutic activity suitable for pharmaceutical or food use selected from ancillary substances known to the person skilled in the art such as for example, diluents, solvents (including water, glycerine, ethyl alcohol), solubilisers, thickeners, sweeteners, flavour enhancement agents, colorants, lubricants, surfactants, antimicrobials, antioxidants, preservatives, pH stabilising buffers and mixtures thereof. Non-limiting examples of such substances are phosphate buffers (for example, dicalcium phosphate), alkali or alkali-earth metal stearate (for example of magnesium), silicon dioxide, mono- and diglycerides of tatty acids, microcrystalline cellulose, hydroxypropyl cellulose, hydroxypropyl methylcellulose, starch or corn starch, natural or artificial flavours (for example, iron oxides).

Said composition of the invention may be a pharmaceutical composition, a composition for medical devices (Medical Device Regulation (EU) 2017/745 (MDR)), a dietary supplement and/or a food for special medical purposes (FSMP).

In the context of the present invention, the expression “subject/s” is used to indicate mammals (animals and humans), preferably human subjects.

The expression “therapeutically effective amount” is used to indicate the amount of mixture or compound or formulation which elicits the biological or medicinal response in a tissue, system or subject which is sought and defined by a person skilled in the art.

Unless otherwise specified, the expression mixture or composition comprises a component in an amount “comprised in a range from x to y” is used to indicate that said component can be present in the composition in all the amounts present in said range, even though not specified, extremes of the range comprised.

EXPERIMENTAL PART—Analytical method for determining phytosterols in the Secale cereale pollen extract (for example, in the product under the trade name Graminex®).

Phytosterols are dosed using a modified version of the Liebermann-Burchard method. Said method is a colorimetric method based on the transformation of phytosterols into dark blue/green-coloured unsaturated polymeric hydrocarbons following treatment with a sulfuric acid/acetic anhydride mixture. The wavelength used is 730 nm, the reaction temperature 36° C. and the reaction time 20 minutes. The method uses stigmasterol as standard. Three solutions of stigmasterol having the same concentration are prepared to this end to obtain three absorbance values. For each sample to be analysed, two separate solutions are prepared measuring them once (two derivatizations). Therefore, the content of phytosterols, such as stigmasterol, is equal to the mean of the phytosterol values from the two measurements.

The titration procedure is reported below in detail.

A) solutions and preparations for analysis

A1) Reagent solution. Transfer 150 ml of acetic acid to a 1 L double-neck flask provided with a 50 ml dropping funnel and a thermometer. Add 300 ml of acetic acid anhydride. Cool the magnetically stirred solution to a temperature below 4° C. under an N₂ atmosphere. Fil the dropping funnel with 50 ml of sulfuric acid and add it to the mixed mixture drop by drop keeping the temperature below 4° C. Remove ice water cooling. Add 10.0 g of sodium sulphate and mix until the material has dissolved. Store the reagent solution at 4° C. in a dark glass bottle.

A2) Preparation of the sample. Weigh exactly 75 mg of material into a 50 ml calibrated flask. Add 1 g of sodium sulphate. Add 40 ml of dichloromethane, dose the flask and mix for 10 minutes. Dilute to volume with dichloromethane and mix thoroughly.

A3) Sample solution. Allow to settle for 10 minutes. Transfer 5 ml of the turbid supernatant into a 20 mi disposable plastic syringe provided with a 0.45μ filter and filter the solution. Prepare 2 sample solutions for each sample to be analysed.

A4) Stigmasterol standard solution. Weigh 10.0 mg of stigmasterol standard (considering the percentage purity) on a weighing beaker and transfer to a 50 mi calibrated flask. Dissolve and dilute to volume with dichloromethane. Prepare 3 stigmasterol standard solution.

A5) Preparing the test (derivatization). Regulate, thermostat the water in the water bath to 36° C. Transfer 2.0 ml of stigmasterol standard solution (A4), 2.0 ml of sample solution (A3) or 2 ml of dichloromethane (white) into a 50 mi Schott glass bottle. Add 4 ml of reagent solution. Mix, seal the bottle and incubate for 10 minutes at 36° C. in a water bath. Filter quickly through a 0.45 μm filter membrane into a glass cuvette and store it in the dark for 10 minutes. Measure the sample 20 minutes after adding the reactive solution A1).

B) Spectrophotometric measurements. Measure the absorbance of standard solutions and sample solution. Verify whether the results obtained from the first and second sample solutions do not differ by more than 5.0% calculated based on the mean value.

The phytosterol content is calculated according to the formula:

(10×Asa×100)/(Ast×75)=content of phytosterols, such as stigmasterol, by percentage.
Ast=absorbance of the standard solution (mean value).
Asa=absorbance of the sample solution.
75=mg of material.
10=mg of stigmasterol, standard.

The phytosterol content is calculated for each of the two sample solutions and a mean value is calculated.

### EXPERIMENTAL PART—EXAMPLES

Table 1 reports an embodiment of the composition of the invention comprising (a), (b), (c) and (d) formulated for oral use in solid form of tablet, granules/powder, wherein said granules or powder are dosed in sachets (in short, dose).

TABLE 1

| Ingredient | mg/dose |
|---|---|
| (a) Secale cereale pollen extract | 500-1000 |
| (hydrophilic:lipophilic = 20:1) | |
| (b) quercetin | 100-200 |
| (c) lipoic acid or a salt thereof | 300-600 |
| (d) tryptophan | 150-300 |
| additives/excipients | q.s. |

Table 2 reports an embodiment of the composition of the invention comprising (a), (b), (c) and (d) formulated for oral use in solid form of tablet, granules/powder, wherein said granules or powder are dosed in sachets (in shot, dose).

TABLE 2

| Ingredient | mg/dose |
|---|---|
| (a) Secale cereale pollen extract | 500-1000 |
| (hydrophilic:lipophilic = 20:1) | |
| (b) quercetin | 75-150 |
| (c) lipoic acid or a salt thereof | 300-600 |
| (d) tryptophan | 100-200 |
| additives/excipients | q.s. |

### Experimental Part II

Study of the antioxidant and anti-inflammatory effects of the composition according to the present invention comprising: a) pollen b) quercetin c) lipoic acid, d) tryptophan by using an in vitro prostate cancer model.

The model is based on the use of PC3 cells, an immortalized cell line of castration-resistant prostate cancer, obtained by isolation from bone metastases, purchased from ATCC, cultured in RPMI at 7.5% FBS. The inflammation model was obtained by treating the cells with LPS (2 μg/ml, O/N). The experimental model used, i.e., prostate cancer immortalized cell line PC3, therefore represents a pathological prostate model.

### Solubility Studies

The individual substances, received in powdered form, were dissolved based on the data reported in prestigious public databases such as PubChem [https://pubchem.ncbi.nlm.nih.gov/].

a) Pollen

Pollen was dissolved in DMSO. The solution was subsequently filtered with 0.22 μm filters to obtain a sterile solution.

b) Quercetin

Quercetin was dissolved in ethanol. The solution was subsequently filtered with 0.22 μm filters to obtain a sterile solution.

c) Lipoic Acid

Lipoic acid was dissolved in methanol. The solution was subsequently filtered with 0.22 μm filters to obtain a sterile solution.

d) Tryptophan

Tryptophan was dissolved in water. The solution was subsequently filtered with 0.22 μm filters to obtain a sterile solution.

Cytotoxicity Studies—MTT Test

Preliminary cytotoxicity studies were conducted on PC3 cels, treated with the individual substances in order to identify the doses to be used for subsequent experiments, as well as the doses to be mixed to verify synergism.

Cells were seeded in 48 well multiwells, at a density of 10000 cells/well. After 48 hours, they were treated with different concentrations of tryptophan, quercetin, lipoic acid and pollen for 24 hours. The range of concentrations selected for each substance was determined based on the data and standard tests known to the person skilled in the art. The MTT test was conducted at the end of the treatment.

Briefly, the growth medium was removed and replaced with phenol red-free medium, containing 0.5 mg/ml MTT (3-(4,5-dimethylthiazol-2-yl)-2,5-diphenyltetrazolium bromide), and the cells were placed in an incubator for one hour. The conversion of yellow MTT to purple formazan by mitochondrial enzymes indicates mitochondrial activity and therefore cell viability. The formazan deposits in the cells were then solubilised with isopropanol, obtaining a homogeneous solution whose absorbance can thus be measured at the spectrophotometer (absorbance at 510 nm).

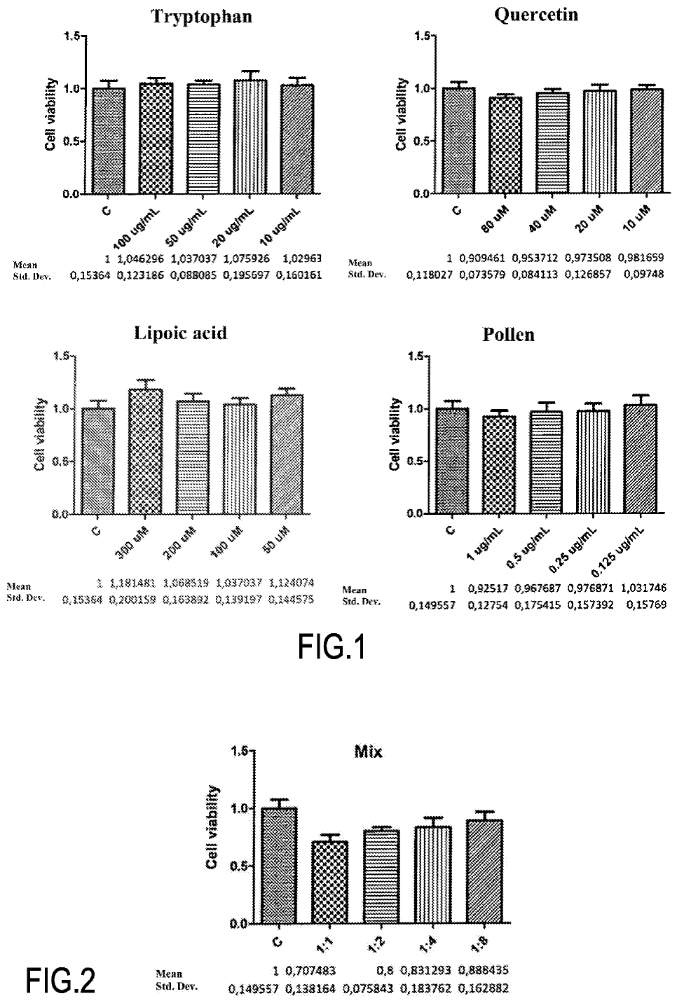

The statistical analysis was conducted using the GraphPad Prism program, using one-way ANOVA, followed by Dunnet tests for multiple comparisons. Cells with a cell viability greater than 70% were considered viable (FIG. 1).

Based on these results, the subsequent experiments were conducted with the following doses (obtained by means of three serial dilutions 1:2):
Tryptophan: 12.5, 25, 50, 100 μg/ml
Quercetin: 10, 20, 40, 80 μM
Lipoic acid: 37.5, 75, 150, 300 μM
Pollen: 0.125, 0.25, 0.5, 1 μg/ml.

### Cytotoxicity—Combination Tests

Similar cytotoxicity tests were thus conducted to check for possible toxicity due to the combination of the substances. The mixture was prepared by combining the maximum doses of each substance a)-d): tryptophan 100 μg/ml, quercetin 80 μM, lipoic acid 300 μM, pollen 1 μg/ml. The subsequent concentrations are obtained by means of three serial dilutions 1:2 (1:2, 1:4, 1:8, respectively). The MTT test was conducted as described above for the individual substances

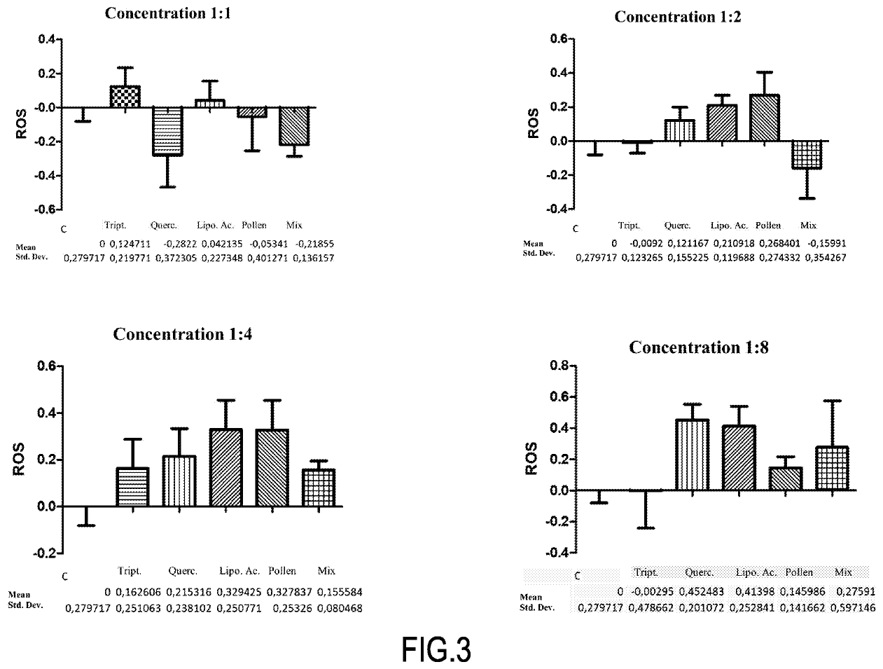

FIG. 2 shows that the combination of the four substances, at each of the concentrations tested, does not exert toxic effects exceeding 30% on PC3 cells.

### Analysis of the Production of Radical Oxygen Species (ROS)

The efficacy of individual substances and the combination thereof in combating the production of radical oxygen species (ROS) already highly produced at baseline in this prostate cancer model was analysed. PC3 cells were seeded in 96-well black multiwells, at a density of 5000 cells each. After 48 hours, a pre-treatment was conducted with different concentrations of the individual substances, a)-d), or of the mixture thereof (according to the present invention). After 24 hours from the treatment, the H2DCFDA (2′,7′-dichlorodihydrolluorescein diacetate) probe was added to the growth medium, which is cleaved by enzymes involved in oxidative stress. The product of this conversion, DCF (dichlorofluorescein), is capable of absorbing light at a λex 492-495 nm, and reemitting at a λem 517-527 nm.

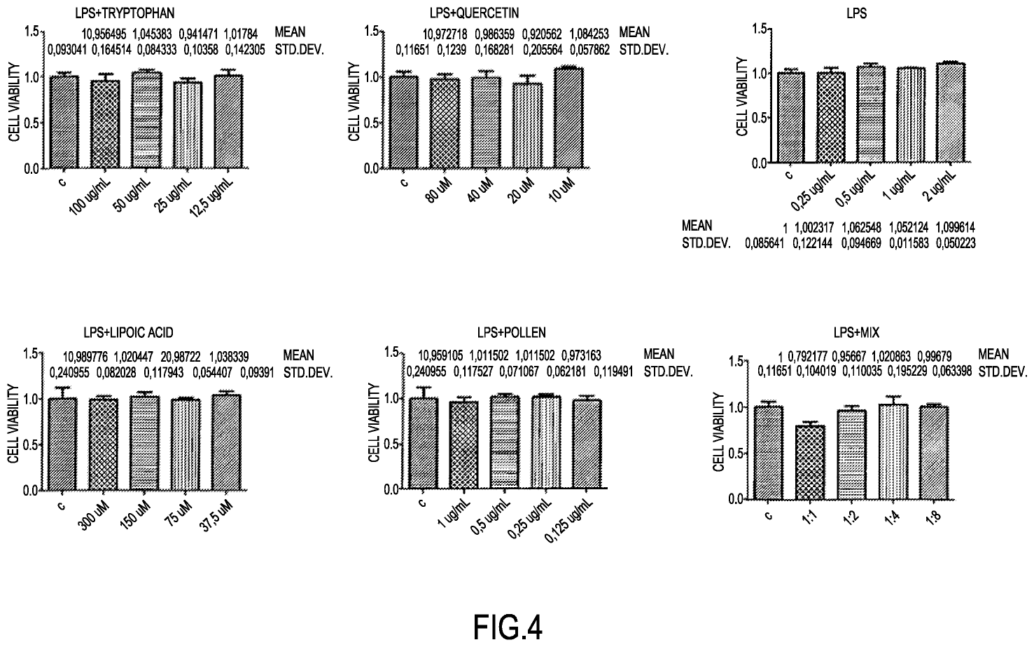

It is interesting to note that under baseline conditions the combination of the active components at the 1:2 dilution shows a higher, synergistic (that is higher than the sum of the effects of the individual active components) antioxidant activity than that of the individual active components at the same dose (FIG. 3). Specifically, while individual active components exert a pro-oxidising action, the combination thereof acts in the opposite way, in this manner inhibiting ROS production. This result is noteworthy given that PC3 cells are characterised, at baseline, by a high intracellular ROS concentration, therefore being representative of a pathological condition, i.e. already at baseline they are characterised by high levels of oxidative stress.

### Cytotoxicity Tests in the Presence of LPS

In order to evaluate the ability of the substances to combat the inflammatory process, the PC3 cells were treated with LPS, a known inflammatory inducer, in the presence or in the absence of the individual substances a)-d) or of the mixture thereof (according to the present invention) To confirm that the concentration of LPS (and the combination thereof with the substances) was not toxic, an MTT test was conducted on the PC3 cells treated according to the following experimental scheme.

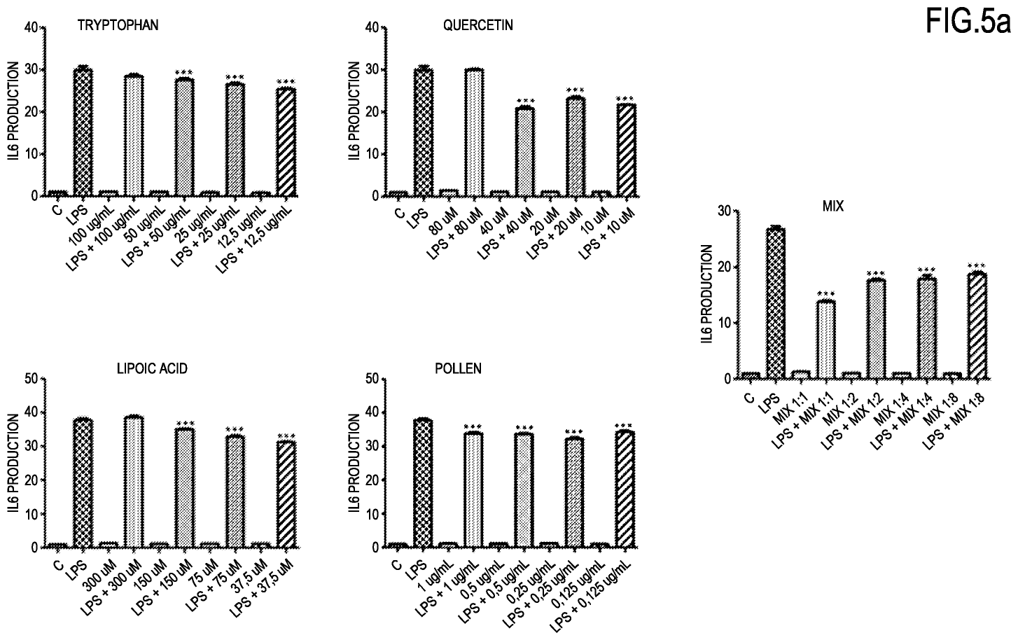

PC3 cells were seeded in 48-well multiwells, at the density of 10,000 cells each, and after 32 hours they were treated with LPS 2 μg/ml O/N, with the aim of inducing the inflammatory process. The subsequent day, the cells were treated with different dosages of the individual substances, or of the combination thereof, for 24 hours. Lastly, the MTT test was conducted as described above. The statistical analysis was conducted using the GraphPad Prism program, using one-way ANOVA, followed by Dunnett's tests for multiple comparisons. FIG. 4 shows that LPS has no toxic effects on PC3 cells, at the dose used. At the same time, it can be observed that the combination thereof with the four substances taken individually or mixed according to the present invention does not induce any toxic effect exceeding 30%.

### Dosage of Inflammation Markers—IL-6

Once the absence of toxicity in the presence of LPS was established, the efficacy of the individual substances, and of the mixture according to the invention, in reducing the production of inflammation mediators was analysed. To this end, PC3 cells were seeded in 6-well multiwells, at a density of 100,000 cells each. After 32 hours, the inflammatory process was induced by treating with LPS 2 μg/ml, O/N. The subsequent day, the cells were treated with different concentrations of the individual substances, or of the combination thereof. After 24 hours of treatment, the conditioned medium of the PC3 cells was harvested, aliquoted, and stored for subsequent analysis. The production of inflammation mediators was evaluated using ELISA tests on the conditioned medium, following the protocols indicated on the respective datasheets. The selected inflammation marker for this analysis is: IL6 (R&D Systems, Cat. No. #DY-206).

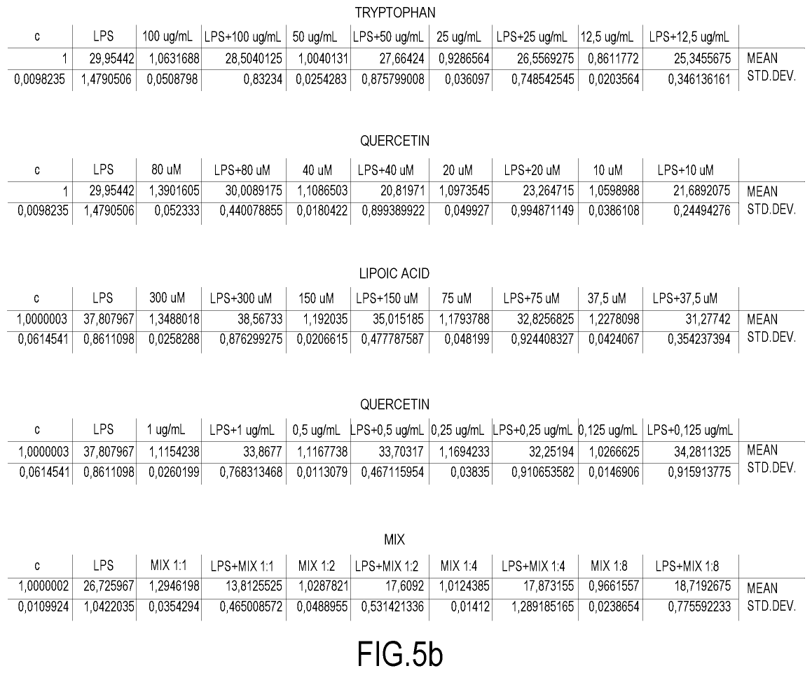

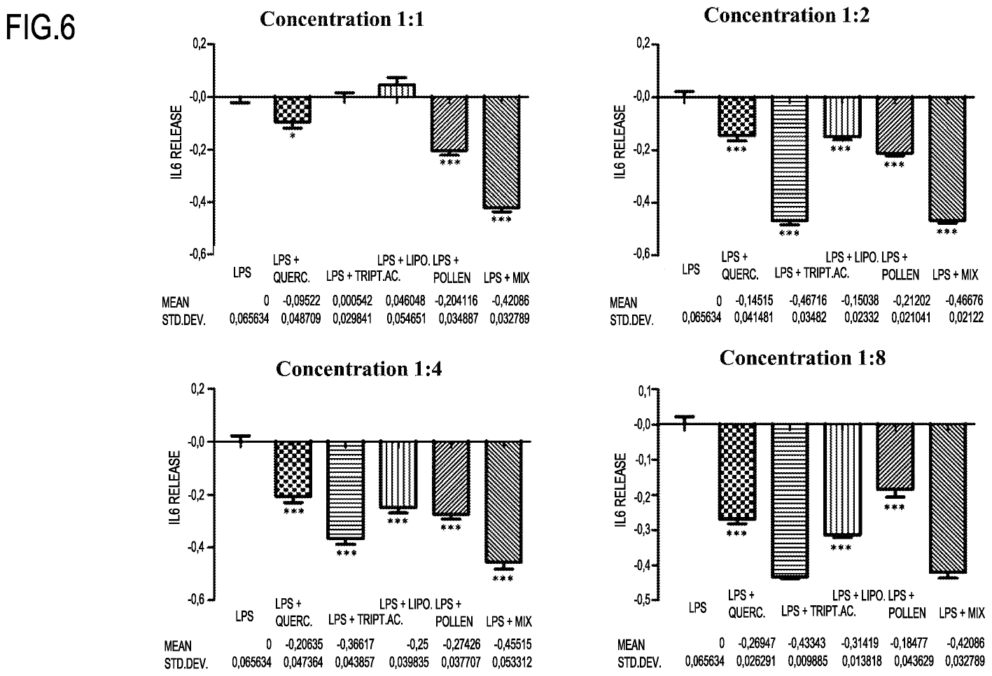

The statistical analysis relating to the results reported in FIGS. 5a and 5b was conducted using the GraphPad Prism program, using one-way ANOVA, followed by Turkey's range test for multiple comparisons. The statistical analysis relating to the results reported in FIG. 6 was conducted using the GraphPad Prism program, using one-way ANOVA, followed by Dunnett's test for multiple comparisons. FIG. 5a shows the dosage of the IL6. As observable, the inflammatory process was efficiently induced by treatment with LPS (positive control), and it is significantly reduced by treatment with both the individual active substances and the combination thereof. Furthermore, it is interesting to note that the combination of the active components at the 1:1 dilution shows a higher and synergistic (that is higher than the sum of the effects of the individual active components) inhibitory activity on the production of IL6 than that of the individual active components at the same dose (FIG. 6).

### CONCLUSIONS

### Cytotoxicity Studies

In conclusion, FIGS. 1, 2 and 4 show that, at the doses used, the active components a)-d), the mixture thereof, and the treatment with the pro-inflammatory stimulus (LPS) does not cause cytotoxicity. This ensures that the results obtained are the expression of the biological activity exerted by the components tested, and that they ae not derived from impaired cell viability. Furthermore, the tested dose range for each active component is physiological, that is it reflects the plasma concentrations that can be obtained following oral intake of this substance.

### Analysis of the Production of Radical Oxygen Species (ROS)

Being a prostate cancer line, and therefore pathological model, PC3 cells are characterised by a high intracellular ROS concentration already under baseline conditions. FIG. 3 shows that the effect of the combination of the active components (mixture according to the invention) has a synergistic effect (that is higher than the sum of the effects of the individual active components), with respect to the administration of the individual components, as regards the reduction of oxidative stress. Specifically, while individual active components exert a pro-oxidising action, the combination thereof acts in the opposite way, in this manner inhibiting ROS production.

FIG. 6 shows the effects of the active components and of the combination thereof on the production of the pro-inflammatory interleukin IL-6 induced by LPS in PC3 cells. Also in this case, it is clear that the combination of the active components (1:1 dilution) shows a higher and synergistic (that is higher than the sum of the effects of the individual active components) inhibitory activity on the production of IL-6 than that of the individual active components at the same dose.

1. A mixture comprising or, alternatively, consisting of:
(a) a Secale cereale (L.) pollen extract,
(b) a quercetin,
(c) a lipoic acid or an acceptable pharmaceutical or food grade salt thereof, and
(d) a tryptophan.

2. The mixture according to claim 1, wherein said (a) Secale cereale (L.) pollen extract, comprises a water-soluble fraction and a liposoluble fraction in a [water-soluble fraction: liposoluble fraction] by weight ratio comprised in the range from 30:1 to 10:1, preferably in a 20±2:1±0.1 by weight ratio, wherein the water-soluble fraction is standardised in amino acids and the liposoluble fraction is standardised in phytosterols.

3. A composition comprising the mixture according to claim 1, and furthermore at least one food or pharmaceutical grade additive and/or excipient.

4. The composition according to claim 3, wherein said composition is formulated for oral use.

5. The composition according to claim 4, wherein said composition formulated for oral use is in solid form, preferably in the form of tablets, capsules, soluble granules or powder, mouth dissolvable granules or powder.

6. The mixture or the composition according to claim 1, wherein a dosing unit or a daily dose unit of said mixture or composition comprises the following amounts:
from 500 mg to 1000 mg of said (a) a Secale cereale (L.) pollen extract, preferably it comprises the water-soluble fraction and the liposoluble fraction in a by weight ratio comprised in the range from 30:1 to 10:1, preferably in a 20±2:1±0.1 by weight ratio;
from 100 mg to 200 mg of said (b) a quercetin;
from 300 mg to 600 mg of said (c) a lipoic acid or a salt thereof; and
from 150 mg to 300 mg of said tryptophan (d).

7. The mixture or the composition according to claim 1 for use as medicament.

8. The mixture or the composition according to claim 1 for use in a method for the preventive and/or curative treatment of a prostate disease or symptom.

9. The mixture or the composition for use according to claim 8, wherein said prostate disease or symptom is selected from the group comprising or, alternatively, consisting of: nonbacterial prostatitis, chronic pelvic pain syndrome, and benign prostatic hyperplasia, and symptoms and/or disorders related therewith.

10. The mixture or the composition for use according to claim 9, wherein said symptoms and/or disorders related with nonbacterial prostatitis, chronic pelvic pain syndrome and benign prostatic hyperplasia are selected from the group comprising or, alternatively, consisting of: constant pain in the scrotum and in the anus or in the central-lower abdomen; pain during or after urination; pain during or after ejaculation; frequent urination, both during the day (pollakiuria) and at night (nocturia); feeling of incomplete bladder emptying after urination; urgency and impossibility of delaying urination; erectile dysfunction and decreased sexual desire.

11. A method for treating a patient for a prostrate disease, comprising administering to a patient a composition comprising:
(a) a Secale cereale (L.) pollen extract,
(b) a quercetin,
(c) a lipoic acid or an acceptable pharmaceutical or food grade salt thereof, and
(d) a tryptophan.

12. The method of claim 11, wherein said wherein said (a) Secale cereale (L.) pollen extract, comprises a water-soluble fraction and a liposoluble fraction in a [water-soluble fraction: liposoluble fraction] by weight ratio comprised in the range from 30:1 to 10:1, wherein the water-soluble fraction is standardised in amino acids and the liposoluble fraction is standardised in phytosterols.

13. The method of claim 11, wherein the method comprises administering a daily dose of:
from 500 mg to 1000 mg of said (a) a Secale cereale (L.) pollen extract;
from 100 mg to 200 mg of said (b) a quercetin;
from 300 mg to 600 mg of said (c) a lipoic acid or a salt thereof; and
from 150 mg to 300 mg of said tryptophan (d);
to the patient.

14. The method of claim 11, wherein the prostate disease is nonbacterial prostatitis, chronic pelvic pain syndrome, or benign prostatic hyperplasia.
